(19) United States
(12) Patent Application Publication
(10) Pub. No.: US 2006/0194759 A1
(43) Pub. Date: Aug. 31, 2006

(54) Topical compositions and methods for treating pain and inflammation

(76) Inventor: Stewart G. Eidelson, Boca Raton, FL (US)

Correspondence Address: Raymond P. Niro;Niro, Scavone, Haller & Niro, Suite 4600, 181 W. Madison, Chicago, IL, 60602, US

(21) Appl. No.: 11/066,863

(22) Filed: Feb. 25, 2005

(51) Int. Cl. A61K 31/737; A61K 31/728; A61K 31/573; A61K 31/045; A61K 31/7008

(52) U.S. Cl. 514/54; 514/171; 514/729; 514/62

(57) ABSTRACT

A topical composition and method for treating pain and inflammation by administering an effective amount of a topical composition comprising an anti-inflammatory steroid such as hydrocortisone, a topical anesthetic such as lidocaine, menthol, and a medically acceptable carrier into which the forgoing are incorporated. A chondroprotective agent can also be added.

### BACKGROUND OF THE INVENTION

1. Field of the Invention

This invention relates to the relief of pain and inflammation, in particular to the control of pain and inflammation by the application of a topical composition containing active ingredients in a suitable carrier for their transport through a patient's skin.

2. Description of the Related Art

Managing pain and inflammation remains a challenge today in medicine. Current treatment options include the oral administration of opioid analgesics such as morphine, codeine and hydrocodone. The oral administration of non-steroidal anti-inflammatory drugs (NSAID) such as aspirin and ketoprofen provides another option in managing pain and inflammation. Despite their effectiveness, the oral use of the above classes of drugs is associated with various adverse events. For instance, opioid analgesics dispose a patient to iatrogenic addiction, where the patient develops a dependency on the potent drug and consequently abuses it. The use of opioid analgesics is also associated with undesired symptoms such as sedation and constipation. Likewise, pain management by the oral administration of non-steroidal anti-inflammatory drugs is associated with irritation to the gastrointestinal tract, gastric bleeding, and ulcer. Other more serious adverse events associated with the oral administration of NSAID's include increased risks in heart attacks and related cardiovascular diseases, as identified in Aleve® and Vioxx®. Thus, many topical compositions containing NSAID's and opioid analgesics, either alone or in combination with other active ingredients, have been developed to by-pass the gastrointestinal tract and therefore mitigate the adverse events associated with oral administration. Such topical compositions also have the additional advantage of acting much faster in relieving pain and inflammation. However, a challenge remains in optimizing and enhancing the therapeutic effects of these topical compositions.

### SUMMARY OF THE INVENTION

The present invention relates to the relief of pain and inflammation by the topical application of a composition comprising a combination of remedies in a carrier composition to enhance their transport through a patient's skin. In particular, the present invention achieves new and unexpected synergistic effects in treating pain and inflammation from the combination of an anti-inflammatory steroid such as hydrocortisone, a topical anesthetic such as lidocaine, and menthol at appropriate concentrations in a medically accepted carrier composition. Chondroprotective agents such as chondroitin sulfate and/or glucosamine can also be added to further enhance the pain-relieving and anti-inflammatory properties of the present invention.

In the present invention, the appropriate concentration of the medically accepted carrier may be the amount effective to facilitate the transmission of the topical compositions of the present invention through the skin. The appropriate concentration of menthol is preferably in an amount from about 1% to about 10% of the composition by weight. The appropriate concentrations of the topical anesthetic such as lidocaine, and the steroidal anti-inflammatory drug such as hydrocortisone are each preferably in amounts from about 1% to about 4% of the composition by weight. The active ingredients of the invention at such concentration ranges will relieve pain and inflammation in an enhanced and synergistic manner. Such benefits may diminish if the concentrations of the individual ingredients are significantly below or significantly above the preferred ranges. For instance, active ingredient concentrations significantly above the preferred ranges may produce unwanted side effects such as the thinning of the epidermis, skin rashes, suppression of the adrenal glands, or excessive desensitization of the skin. On the other hand, active ingredient concentrations significantly below the preferred ranges may not be in sufficient amounts to produce the desired effects of the present invention.

### DETAILED DESCRIPTION OF THE INVENTION

The present invention relates to topical compositions that can contain a combination of an anti-inflammatory steroid such as hydrocortisone, a topical anesthetic such as lidocaine, menthol, and a medically acceptable carrier at appropriate concentrations to relieve pain and inflammation. In another embodiment, the above compositions may contain one or more chondroprotective agents such as chondroitin sulfate and/or glucosamine at therapeutically effective concentrations.

1. Anti-Inflammatory Steroid

Many steroids have potent anti-inflammatory properties and are used to treat a variety of conditions such as arthritis, colitis, asthma, bronchitis, certain skin rashes, and allergic or inflammatory conditions of the nose and eyes. Unlike NSAID's that reduce inflammation by inhibiting the biosynthesis of prostaglandins, steroidal anti-inflammatory agents reduce inflammation by inducing protein synthesis via gene expression. A desirable anti-inflammatory steroid for the present invention is hydrocortisone. Hydrocortisone is a natural corticosteroid produced by the adrenal glands. Other steroids suitable for the present invention include alcometasone, clocortolone, dexamethasone, hydrocortisone 21-acetate, prednisone, hydrocortisone 17-valerate, hydrocortisone 17-butyrate, betamethasone valerate, triamcinolone acetonide, flucinonide, desonide, flucinolone acetonide, dexamethasone 21-phosphate, prednisolone, prednisolone 21-phosphate, haloprednone, cortisone acetate, hydrocortisone cyclopentylpropionate, cortodoxone, flucetonide, fludrocortisone acetate, flurandrenolone acetonide, medrysone, amcinafal, amcinafide, betamethasone, betamethasone benzoate, chloroprednisone acetate, clocortolone acetate, descinolone acetonide, desoximetasone, dichlorisone acetate, difluprednate, flucloronide, flumethasone, flumethasone pivalate, flunisolide acetate, flucortolone, fluorometholone, fluperolone acetate, fluprednisolone, fluprednisolone valerate, meprednisone, methyl prednisolone, paramethasone acetate, prednisolamate, prednival, triamcinolone, triamcinolone hexacetonide, cortivazol, formocortal, nivazol or methylprednisone, beclomethasone 17,21-dipropionate, betamethasone 17-valerate, betamethasone 17,21-dipropionate, clobetasol 17-propionate, clobetasone 17-butyrate, mixtures thereof, and equivalents thereof.

In the present invention, the steroidal anti-inflammatory compound such as hydrocortisone is preferably present in an amount from about 1% to about 4%, preferably from about 1% to about 2%, and more preferably approximately 1% of the composition by weight. The steroidal anti-inflammatory compound at the above concentration ranges, in combination with the other active ingredients of the present invention at appropriate concentrations, enhances and synergizes the relief of pain and inflammation. Steroidal anti-inflammatory drug concentrations significantly below 1% may be too low to produce such effects. The therapeutic effects of the present invention may also be affected at steroidal concentrations significantly above 4%. Continued and prolonged usage at such concentrations may enhance a patient's disposition to various skin conditions such as drying, cracking, irritation, suppression of the adrenal glands, Cushing's syndrome, excessive fluid retention, dermatitis, allergic reactions, thinning of the skin, and susceptibility to infections.

2. Topical Anesthetic

Topical anesthetics operate by desensitizing and blocking pain pathways at the skin level. The preferred topical anesthetic for the present invention is lidocaine. Other topical anesthetics suitable for the present invention include but are not limited to benzocaine, butamben, dibucaine, propoxycaine, procaine, mepivacaine, bupivacaine, pramoxine, tetracaine, mixtures thereof and equivalents thereof. The topical anesthetic such as lidocaine in the present invention is preferably in an amount from about 1% to about 4%, preferably from about 2% to about 4%, and more preferably approximately 2% of the composition by weight. Such concentration ranges will ensure that appropriate amounts of the topical anesthetic penetrates the epidermis and produces the enhanced and synergistic effects of the present invention in relieving pain and inflammation. Topical anesthetic concentrations significantly below 1% may be insufficient for producing such effects. On the other hand, the prolonged usage of topical anesthetics on the skin at concentrations significantly above 4% may lead to excessive dermal desensitization, the swelling and itching of the skin, the development of skin rashes, or burning sensations on the skin.

3. Menthol

Menthol is a compound obtained from peppermint oil with local anesthetic and counterirritant qualities. Menthol is readily absorbed by the skin, providing a temporary cooling effect to limit swelling, decrease pain, and relax muscles. In addition, menthol enhances the transport of chemicals through the skin by enhancing skin penetration and absorption. In the present invention, menthol will preferably be present in an amount from about 1% to about 10%, preferably from about 2% to about 5%, and more preferably approximately 3% of the composition by weight. Menthol concentrations significantly below 1% may not be sufficient to produce the desired synergistic and enhanced effects of the present invention in relieving pain and inflammation. Menthol concentrations significantly above 10% may mitigate such effects by producing excessive and undesired cooling sensations on the skin after prolonged usage. Thus, it is desirable for the compositions of the present invention to use menthol in the preferred concentration ranges.

4. Chondroprotective Agents

Chondroprotective agents are integral components of connective tissues such as the articular cartilage. These agents have anti-inflammatory and pain relieving properties in patients suffering from osteoarthritis and other inflammatory conditions. It is hypothesized that chondroprotective agents have such effects by interfering with the inflammatory cascade, inhibiting cartilage-degrading enzymes, stimulating the production of new matrix and structural proteins, improving the quality of synovial fluid to enhance cartilage nutrition and lubrication, stimulating the production of free radical scavenging enzymes, and improving blood flow to joint tissues. Examples of chondroprotective agents suitable for the present invention include but are not limited to chondroitin sulfate, glucosamine, N-acetyl glucosamine, polysulfated glycosaminoglycan, hyaluronic acid, pentosan polysulfate, and derivatives thereof. Chondroitin sulfate and glucosamine are the best studied and therefore the preferred chondroprotective agents for use in the present invention. It is also desirable that glucosamine and chondroitin sulfate be used together in the same composition due to enhanced and synergistic effects. In the present invention, chondroprotective agents will be present in therapeutically effect amounts. Such concentration ranges may span from about 0.5% to about 95%, preferably from about 5% to about 40%, and more preferably about 30% by weight of the composition.

5. Medically Acceptable Carriers and Additives

The active components of the topical compositions of the present invention can be suspended in a suitable carrier that promotes their rapid transport through a patient's skin. Medically acceptable carriers of the present invention are chosen so that they are generally compatible with the individual components of the present invention and do not interfere significantly with their transport through a patient's skin. The carrier composition may be an individual compound or a plurality of compounds. Examples of carrier compounds that may be used in the compositions of the present invention include but are not limited to water (preferably deionized), mineral oil, salicylic acid, methylsulfonylmethane (MSM), jojoba oil, alcohol (e.g., ethanol, isopropanol), a mono- or polyglycol (e.g., ethylene glycol, propylene glycol, polyethylene glycol, polyethylene glycol-8 stearate, polyoxyalkylene derivatives, propylene glycol), fatty acid esters (e.g., alkyl stearates, oleates, linoleates, isopropyl palmitate) or other organic compounds or polymers such as polyacrylamides, dimethylsulfoxide, dimethylformamide, dimethylacetamide, 1,2,6-hexanetriol, butanediol, and equivalents thereof.

In addition to the carrier composition and active ingredients, the topical compositions of the present invention may include additives such as anti-oxidants (e.g. butylated hydroxytoluene, butylated hydroxyanisole, propyl gallate), perfumes (e.g. rosemary oil), colorants, moisturizers and emollients (e.g. sunflower oil, jojoba oil, isopropyl palmitate). Agents such as peppermint oil may also be added to the compositions of the present invention to enhance their cooling effects. Other suitable additives include preservatives for maintaining the chemical structure and stability of the active ingredients in the compositions of the present invention. Preservatives may include anti-microbial agents and anti-fungal agents such as propylene glycol, methyl paraben, propyl paraben, and diazodinyl urea. An example of a commercially available product that contains a blend of such agents and is suitable for the present invention is Germaben® II by Nature Bath (North Ridgeville, Ohio).

Emulsifying agents can also be added to the topical compositions of the present invention. Examples of emulsifying agents suitable for the present invention include but are not limited to C13-C14 isoparaffin, laureth-7, polyacrylamides, and polyglycols.

Like the carrier composition, the additional components of the present invention are chosen so that they are generally compatible with the active ingredients in the compositions of the present invention and do not significantly hinder their absorption through a patient's skin.

6. Various Forms of the Topical Composition of the Present Invention

The topical compositions of the present invention may be in the form of a liquid, lotion, ointment, cream, salve, spray, gel, or other equivalent forms. Such forms of the topical composition are achieved by using conventional methods used in the art.

The degree of viscosity of topical compositions in the present invention may be controlled by the use of suitable gelling and/or thickening agents that include but are not limited to oils, alcohols, fatty acids, various polymers, and mixtures thereof. Specific examples of thickening and/or gelling agents suitable for the present invention include but are not limited to peanut oil, castor oil, peppermint oil, rosemary oil, jojoba oil, sunflower oil, aluminum stearate, cetostearyl alcohol, propylene glycol, polyethylene glycols, polyacrylamides, hydroxypropyl cellulose, hydroxypropyl methyl cellulose, hydroxyethyl cellulose, carboxyl vinyl polymers, C13-C14 isoparaffin, liquid paraffin, soft paraffin, laureth-7, woolfat, hydrogenated lanolin, beeswax, and combinations thereof. These agents are added to the components of the present invention in any order and agitated in a container to homogeneity.

Like the additives and carrier compositions, the agents used to achieve the various forms of the present invention are chosen so that they are generally compatible with the active components in the topical compositions of the present invention and do not significantly hinder their absorption through a patient's skin.

It is also to be understood that many of the ingredients suitable for the present invention can serve more than one function. For instance, polyacrylamides and polyglycols can serve both as carriers and emulsifying agents in a composition. Polyglycols can also serve as preservatives. Likewise, isopropyl palmitate and jojoba oil in a composition can serve as emollients, moisturizers and carriers. Similarly, laureth-7 and C13-C14 isoparaffin can serve as emulsifiers, gelling agents, and thickening agents.

7. Applications of the Disclosed Compositions

The topical compositions of the present invention may be used to alleviate pain and inflammation, including but not limited to pain and inflammation associated with the muscular system, the skeletal system, the nervous system, and the epidermis. The compositions herein may also be used to alleviate pain and inflammation associated with connective tissues.

Examples of conditions that can be treated with the compositions of the present invention include but are not limited to pain and inflammation associated with arthritis and cancer. More specific examples include but are not limited to osteoarthritis, rheumatoid arthritis, psoriatic arthritis, acute gouty arthritis, ankylosing spondylitis, juvenile arthritis, arthritis associated with an infection, hematomas, sarcomas, osteosarcomas, metastic cancer, breast cancer, and prostrate cancer. The compositions of the present invention may also be used to alleviate pain and inflammation associated with the joints, head, neck, face, shoulder, and back. Specific examples include myofascial pain and migraine headaches. Other non-limiting examples of conditions associated with pain and inflammation that may be treated with the compositions of the present invention include muscle tension, pinched nerves, strains, sprains, spasms, whiplashes, systemic lupus, psoriasis, Crohn's disease, dermatitis herpeptiformis, temporal mandibular joint syndrome, carpal tunnel syndrome, and fibromyalgia.

It is desirable that the topical compositions of the present invention be used to treat pain and inflammation in human patients. However, the topical compositions of the present invention may also be used to treat pain and inflammation in animals.

8. Methods of Using the Disclosed Compositions

The topical compositions of the present invention may be used to treat pain and inflammation by various methods. In one embodiment, the composition is poured on to the area on the skin affected with pain and inflammation. The area is then massaged or rubbed until the composition is distributed evenly or disappears. The process can be repeated several times, preferably 5-6 times and more preferably 1-2 times in a day. In another method, the topical composition is applied to a dermal patch, which is then mounted onto the affected area of the skin for 30 minutes to several hours. In another embodiment, the topical composition of the present invention is delivered to the affected area using iontophoresis. In this method, the topical composition is placed in a container or a patch that is connected to an electrode. The container is then placed on the affected area, and the electrode is activated. This leads to the generation of a current that delivers the topical composition through the skin by electrical repulsion.

Other suitable methods for delivering the topical compositions of the present invention through the skin include phonophoresis and cellophane wrapping. In phonophoresis, the topical composition is first applied to the affected area on the skin. An ultrasound apparatus is then placed on the affected area. Once activated, the apparatus delivers the composition through the skin by ultrasonic energy. In cellophane wrapping, the composition is applied to the affected area and wrapped with a cellophane film anywhere from several minutes to several hours.

It is to be understood that there are many other ways of using the compositions of the present invention. The above examples simply illustrate various embodiments and do not limit the scope of the present invention.

### DESCRIPTION OF THE PREFERRED EMBODIMENTS

The following antidotal examples illustrate the invention. The scope of the present invention is not limited these or any other examples.

### Example 1

### Preparation of a Topical Composition

Sunflower oil, jojoba oil, isopropyl palmitate and polyethylene glycol-8 stearate are mixed in an appropriate stainless steel tank, blended at high speed and heated. A separate mixture of deionized water, methylsulfonylmethane (MSM), lidocaine and hydrocortisone are also prepared in a similar fashion. The two compositions are then combined, agitated and further heated. Next, menthol, rosemary oil and peppermint oil are added to the composition and agitated to homogeneity. The anti-microbial preservative blend, Germaben® II, which contains propylene glycol, diazolidinyl urea, methylparaben and propylparaben is then added. This is followed by the addition of an emulsifier blend containing polyacrylamide, C 13-14 isoparaffin and laureth-7. After blending the mixture to homogeneity, the composition is assayed for menthol, lidocaine and hydrocortisone. The final concentrations of menthol, lidocaine, and hydrocortisone in the composition will be 3%, 2%, and 1% by weight, respectively.

### Example 2

### Treating Pain in Human Patients with the Topical Composition of Example 1

Case history 1. The composition of Example 1 was applied to the area of the skin associated with back pain on a patient. On a 0-10 scale, where 10 is the worst pain possible and 0 is no pain, the patient's pain level decreased from 6 before treatment to 3 two hours after treatment.

Case history 2. The composition of Example 1 was applied to the area of the skin associated with wrist pain on a patient. The patient's pain level decreased from level 5 before topical application to level 3 two hours after treatment.

Case History 3. The composition of Example 1 was applied to the area of the skin associated with myofascial pain on a patient. The pain level decreased from level 5 before topical application to level 2 two hours after treatment.

Case History 4. The composition of Example 1 was applied to the area of the skin associated with neck pain on a patient. The pain level decreased from level 6 before topical application to level 2 two hours after treatment.

The above tests and results are antidotal reports only. They are not intended to be exhaustive or complete. Nevertheless, the studies demonstrate the effectiveness of the compositions of the present invention in relieving pain. The above studies are summarized in

TABLE 1
TABLE 1

| | | Pain level | Pain level |
|---|---|---|---|
| Case | | Before topical | 2 hours after |
| History | Condition | application | topical application |
| 1 | Back pain | 6 | 3 |
| 2 | Wrist pain | 5 | 3 |
| 3 | Myofascial pain | 5 | 2 |
| 4 | Neck pain | 6 | 2 |

It is to be understood that the individual components of the present invention, either alone or in association with other ingredients, may not act as effectively as when in the compositions of the present invention at the appropriate concentrations. It will be evident that there are numerous embodiments of the present invention which, while not expressly described above, are clearly within the scope and spirit of the invention. The above description is therefore intended to be exemplary only and the scope of the invention is to be determined solely by the appended claims.

What is claimed:

1- A topical composition for treating pain and inflammation comprising:
(a) about 1% to about 4% by weight of an anti-inflammatory steroid;
(b) about 1% to about 4% by weight of a topical anesthetic;
(c) about 1% to about 10% by weight of menthol; and
(d) a medically acceptable carrier at a concentration effective to transport said composition through skin.

2- The topical composition of claim 1 further comprising a chondroprotective agent.

3- The topical composition of claim 2 wherein the chondroprotective agent is selected from the group consisting of chondroitin sulfate, glucosamine, polysulfated glycosaminoglycan, hyaluronic acid, pentosan polysulfate, and mixtures thereof.

4- The topical composition of claim 2 wherein the concentration range of the chondroprotective agent is from about 0.5% to about 95% by weight of the composition.

5- The topical composition of claim 1 wherein the anti-inflammatory steroid is selected from the group consisting of alcometasone, clocortolone, dexamethasone, hydrocortisone, hydrocortisone 21-acetate, prednisone, hydrocortisone 17-valerate, hydrocortisone 17-butyrate, betamethasone valerate, triamcinolone acetonide, flucinonide, desonide, flucinolone acetonide, dexamethasone 21-phosphate, prednisolone, prednisolone 21-phosphate, haloprednone, cortisone acetate, hydrocortisone cyclopentylpropionate, cortodoxone, flucetonide, fludrocortisone acetate, flurandrenolone acetonide, medrysone, amcinafal, amcinafide, betamethasone, betamethasone benzoate, chloroprednisone acetate, clocortolone acetate, descinolone acetonide, desoximetasone, dichlorisone acetate, difluprednate, flucloronide, flumethasone, flumethasone pivalate, flunisolide acetate, flucortolone, fluorometholone, fluperolone acetate, fluprednisolone, fluprednisolone valerate, meprednisone, methyl prednisolone, paramethasone acetate, prednisolamate, prednival, triamcinolone, triamcinolone hexacetonide, cortivazol, formocortal, nivazol or methylprednisone, beclomethasone 17,21-dipropionate, betamethasone 17-valerate, betamethasone 17,21-dipropionate, clobetasol 17-propionate, clobetasone 17-butyrate, and mixtures thereof.

6- The topical composition of claim 1 wherein the topical anesthetic is selected from the group consisting of benzocaine, butamben, dibucaine, lidocaine, propoxycaine, procaine, mepivacaine, bupivacaine, pramoxine or tetracaine, and mixtures thereof.

7- The topical composition of claim 1 wherein the medically acceptable carrier is selected from the group consisting of water, mineral oil, salicylic acid, methylsulfonylmethane, jojoba oil, alcohol, ethylene glycol, propylene glycol, polyethylene glycol, polyethylene glycol-8 stearate, alkyl stearates, oleates, linoleates, isopropyl palmitate, polyacrylamides, dimethylsulfoxide, dimethylformamide, dimethylacetamide, 1,2,6-hexanetriol, butanediol, and mixtures thereof.

8- The topical composition of claim 1 further comprising anti-oxidants, perfumes, colorants, moisturizers, emollients, anti-fungal agents, anti-microbial agents, preservatives, emulsifying agents, thickening agents, gelling agents, and other conventional additives known to be effective and suitable for topical applications.

9- The topical composition of claim 1, wherein the composition is in the form of a liquid, lotion, ointment, cream, gel, salve, spray, aerosol, or equivalents thereof.

10- A topical composition for treating pain and inflammation comprising:
(a) about 1% to about 4% by weight of an anti-inflammatory steroid;
(b) about 1% to about 4% by weight of a topical anesthetic;
(c) about 1% to about 10% by weight of menthol;
(d) at least one chondroprotective agent; and
(e) a medically acceptable carrier at a concentration effective to transport said composition through skin.

11- The topical composition of claim 10 wherein the chondroprotective agent is selected from the group consisting of chondroitin sulfate, glucosamine, polysulfated glycosaminoglycan, hyaluronic acid, pentosan polysulfate, and mixtures thereof.

12- The topical composition of claim 10 wherein the concentration range of the chondroprotective agent is from about 0.5% to about 95% by weight of the composition.

13- The topical composition of claim 10 wherein the anti-inflammatory steroid is selected from the group consisting of alcometasone, clocortolone, dexamethasone, hydrocortisone, hydrocortisone 21-acetate, prednisone, hydrocortisone 17-valerate, hydrocortisone 17-butyrate, betamethasone valerate, triamcinolone acetonide, flucinonide, desonide, flucinolone acetonide, dexamethasone 21-phosphate, prednisolone, prednisolone 21-phosphate, haloprednone, cortisone acetate, hydrocortisone cyclopentylpropionate, cortodoxone, flucetonide, fludrocortisone acetate, flurandrenolone acetonide, medrysone, amcinafal, amcinafide, betamethasone, betamethasone benzoate, chloroprednisone acetate, clocortolone acetate, descinolone acetonide, desoximetasone, dichlorisone acetate, difluprednate, flucloronide, flumethasone, flumethasone pivalate, flunisolide acetate, flucortolone, fluorometholone, fluperolone acetate, fluprednisolone, fluprednisolone valerate, meprednisone, methyl prednisolone, paramethasone acetate, prednisolamate, prednival, triamcinolone, triamcinolone hexacetonide, cortivazol, formocortal, nivazol or methylprednisone, beclomethasone 17,21-dipropionate, betamethasone 17-valerate, betamethasone 17,21-dipropionate, clobetasol 17-propionate, clobetasone 17-butyrate, and mixtures thereof.

14- The topical composition of claim 10 wherein the topical anesthetic is selected from the group consisting of benzocaine, butamben, dibucaine, lidocaine, propoxycaine, procaine, mepivacaine, bupivacaine, pramoxine or tetracaine, and mixtures thereof.

15- The topical composition of claim 10 wherein the medically acceptable carrier is selected from the group consisting of water, mineral oil, salicylic acid, methylsulfonylmethane, jojoba oil, alcohol, ethylene glycol, propylene glycol, polyethylene glycol, polyethylene glycol-8 stearate, alkyl stearates, oleates, linoleates, isopropyl palmitate, polyacrylamides, dimethylsulfoxide, dimethylformamide, dimethylacetamide, 1,2,6-hexanetriol, butanediol, and mixtures thereof.

16- The topical composition of claim 10 further comprising anti-oxidants, perfumes, colorants, moisturizers, emollients, anti-fungal agents, anti-microbial agents, preservatives, emulsifying agents, thickening agents, gelling agents, and other conventional additives known to be effective and suitable for topical applications.

17- The topical composition of claim 10, wherein the composition is in the form of a liquid, lotion, ointment, cream, gel, salve, spray, aerosol, or equivalents thereof.

18- A method for treating pain and inflammation by administering an effective amount of a composition comprising:
(a) about 1% to about 4% by weight of an anti-inflammatory steroid;
(b) about 1% to about 4% by weight of a topical anesthetic;
(c) about 1% to about 10% by weight of menthol; and
(d) a medically acceptable carrier at a concentration effective to transport said composition through skin.

19- The method of claim 18 wherein the composition further comprises a chondroprotective agent.

20- The method of claim 18 wherein the chondroprotective agent is selected from the group consisting of chondroitin sulfate, glucosamine, polysulfated glycosaminoglycan, hyaluronic acid, pentosan polysulfate, and mixtures thereof.

21- The method of claim 18 wherein the concentration range of the chondroprotective agent is from about 0.5% to about 95% by weight of the composition.

22- The method of claim of claim 18 wherein the topical composition is applied by direct application, dermal patches, cellophane wrapping, iontophoresis, phonophoresis, or equivalent methods thereof.

23- The method of claim 18 wherein the composition is used to treat pain and inflammation associated with the muscular system, the skeletal system, the nervous system, the epidermis, and connective tissues.

24- The method of claim 18 wherein the composition is used to treat pain and inflammation associated with muscles, joints, nerves, the neck, the shoulders, the back, preoperative and postoperative treatments, bone injuries, arthritis, and cancer.

25- A method for treating pain and inflammation by administering an effective amount of a composition comprising:
(a) about 1% to about 4% by weight of an anti-inflammatory steroid;
(b) about 1% to about 4% by weight of a topical anesthetic;
(c) about 1% to about 10% by weight of menthol;
(d) at least one chondroprotective agent; and
(e) a medically acceptable carrier at a concentration effective to transport said composition through skin.

26- The method of claim 25 wherein the topical composition is applied by direct application, dermal patches, cellophane wrapping, iontophoresis, phonophoresis, or equivalent methods thereof.

27- The method of claim 25 wherein the composition is used to treat pain and inflammation associated with the muscular system, the skeletal system, the nervous system, the epidermis, and connective tissues.

28- The method of claim 25 wherein the composition is used to treat pain and inflammation associated with muscles, joints, nerves, the neck, the shoulders, the back, preoperative and postoperative treatments, bone injuries, and arthritis.
